(19) United States
(12) Patent Application Publication
(10) Pub. No.: US 2011/0306768 A1
(43) Pub. Date: Dec. 15, 2011

(54) PROCESSES FOR THE MANUFACTURE OF 3--PYRIDINE, 4-METHYL-3-METHYLTHIO-5-(3-PYRIDYL)-L,2,4-TRIAZOLE, AND (1R)-1-[2-(3-METHYLPHENYL)-2H-TETRAZOL-5-YL]ETHANOL

(75) Inventors: Jones Elfyn, Sodertalje (SE); Ulf G. Larsson, Sodertalje (SE)

(73) Assignee: ASTRAZENECA AB, Sodertalje (SE)

(21) Appl. No.: 13/140,454

(22) PCT Filed: Dec. 11, 2009
(86) PCT No.: PCT/SE09/51406

§ 371 Date: Sep. 1, 2011

Related U.S. Application Data
(60) Provisional application No. 61/138,749, filed on Dec. 18, 2008.

(51) Int. Cl. C07D 401/14; C07D 257/04; C07D 401/04

(52) U.S. Cl. 546/272.4; 548/252

(57) ABSTRACT

The present invention provides a process for the manufacture of the compound 3-{4-methyl-5-[(1R)-1-(2-(3-methylphenyl-2H-tetrazol-5-yl)-ethoxy]-4H-[1,2,4]triazol-3-yl}-pyridine of formula 14 wherein a) the compound 3-(5-methane-sulfonyl-4-methyl-4H-1,2,4-triazol-3-yl)-pyridine of formula 6 and the compound (1R)-[2-(3-methylphenyl)-2H-tetrazol-5-yl]ethanol of formula 13 are dissolved in an aprotic solvent, whereafter an alkoxide base is added, providing the compound of formula 14. The invention also provides methods for manufacturing 3-(5-methanesulfonyl-4-methyl-4H-1,2,4-triazol-3-yl)-pyridine and (1R)-1-[2-(3-methylphenyl)-2H-tetrazol-5-yl]ethanol.

### FIELD OF THE INVENTION

The present invention relates to a new process for large-scale production of 3-{4-methyl-5-[(1R)-1-(2-(3-methylphenyl-2H-tetrazol-5-yl)-ethoxy]-4H-[1,2,4]triazol-3-yl}-pyridine, which is a pharmacologically useful mGluR5 antagonist. The present invention also provides new processes for large-scale production of 3-(5-methanesulfonyl-4-methyl-4H-1,2,4-triazol-3-yl)-pyridine as well as (1R)-1-[2-(3-methylphenyl)-2H-tetrazol-5-yl]ethanol, which compounds are intermediates in the process of producing 3-{4-methyl-5-[(1R)-1-(2-(3-methylphenyl-2H-tetrazol-5-yl)-ethoxy]-4H-[1,2,4]triazol-3-yl}-pyridine.

### TECHNICAL BACKGROUND

3-{4-Methyl-5-[(1R)-1-(2-(3-methylphenyl-2H-tetrazol-5-yl)-ethoxy]-4H-[1,2,4]triazol-3-yl}-pyridine is an antagonist of the mGluR5 receptor. Accordingly, this compound is expected to be well suited for treatment of mGluR5 receptor-mediated disorders, such as neurological disorders, psychiatric disorders, gastrointestinal disorders, and chronic and acute pain disorders. This and similar compounds are disclosed in WO2009/051556 and WO2005/080356. Furthermore, WO2005/080356 describes a three-step process of producing 3-(5-methanesulfonyl-4-methyl-4H-1,2,4-triazol-3-yl)-pyridine. WO2009/051556 discloses a six-step process of manufacturing (1R)-1-[2-(3-methylphenyl)-2H-tetrazol-5-yl]ethanol as well as synthesis of the final product 3-{4-methyl-5-[(1R)-1-(2-(3-methylphenyl-2H-tetrazol-5-yl)-ethoxy]-4H-[1,2,4]triazol-3-yl}-pyridine.

The processes of WO2009/051556 and WO2005/080356 are complicated multi-step processes that are suitable for laboratory scale. Accordingly, there is a need for an improved process, which is possible to carry out in larger scale, and which ideally is simple, cost effective, and without harmful impact on the environment.

### SUMMARY OF THE INVENTION

In a first aspect, the invention provides a process for the manufacture of the compound 3-{4-methyl-5-[(1R)-1-(2-(3-methylphenyl-2H-tetrazol-5-yl)-ethoxy]-4H-[1,2,4]triazol-3-yl}-pyridine of formula 14

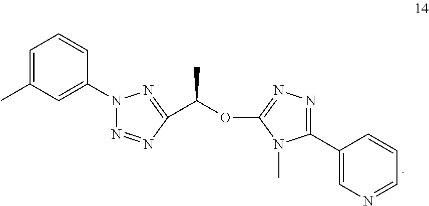

wherein
a) the compound 3-(5-methanesulfonyl-4-methyl-4H-1,2,4-triazol-3-yl)-pyridine of formula 6

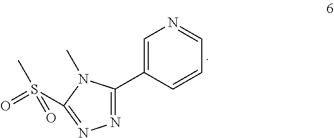

and the compound (1R)-1-[2-(3-methylphenyl)-2H-tetrazol-5-yl]ethanol of formula 13

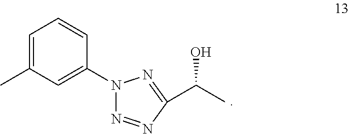

are dissolved in an aprotic solvent, whereafter a base is added, providing the compound of formula 14.

Examples of an aprotic solvent that may be useful in accordance with the invention, is tetrahydrofuran, 2-methyltetrahydrofuran, DMSO, acetonitrile, sulfolan or isopropyl acetate.

Examples of a base that may be useful in accordance with the invention is an alkoxide base such as lithium-, sodium- or potassium tert-butoxide, lithium-, sodium- or potassium tert-amylate, or a hydride base such as sodium or potassium hydride.

Preferably, 3-{4-methyl-5-[(1R)-1-(2-(3-methylphenyl-2H-tetrazol-5-yl)-ethoxy]-4H-[1,2,4]triazol-3-yl}-pyridine is purified by crystallization from isopropyl acetate.

In a second aspect, the invention provides a process for the manufacture of the compound 4-methyl-3-methylthio-5-(3-pyridyl)-1,2,4-triazole of formula 5

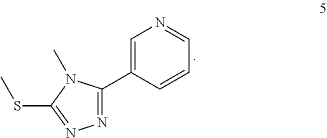

comprising the steps of:
i) dissolving nicotinic acid hydrazide in a first solvent;
ii) adding an isocyanate to the solution of step i) until complete conversion is obtained;
iii) adding a base to the resulting mixture of step ii), providing the compound 4-methyl-5-pyridin-3-yl-2,4-dihydro-3H-1,2,4-triazole-3-thione of formula 4

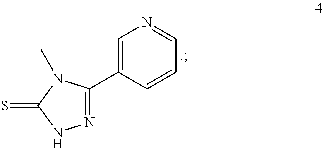

iv) charging methyl iodide to the reaction mixture of step iii);
wherein steps i)-v) are carried out without intermediate isolation;
providing the compound 4-methyl-3-methylthio-5-(3-pyridyl)-1,2,4-triazole of formula 5.

Preferably, the first solvent is chosen from the group of n-butanol and water.

Preferably, the isocyanate is methyl isocyanate.

Preferably, the base is chosen from the group of sodium hydroxide, potassium hydroxide and tributylamine.

In a particularly preferred embodiment, the first solvent is n-butanol, the isocyanate is methyl isocyanate and the base is tributylamine.

In a preferred embodiment, the following steps are carried out after step v) above:

vi) dissolving the compound 4-methyl-3-methylthio-5-(3-pyridyl)-1,2,4-triazole of formula 5 in an acid aqueous solution;
vii) adding a tungstate such as sodium tungstate dihydrate and allowing said tungstate to react with said 4-methyl-3-methylthio-5-(3-pyridyl)-1,2,4-triazole of formula 5;
viii) increasing pH of the reaction resulting in precipitation of the compound 3-(5-methanesulfonyl-4-methyl-4H-1,2,4-triazol-3-yl)-pyridine of formula 4; which is thereafter recovered.

In a preferred embodiment, said acid aqueous solution is a diluted sulphuric acid solution.

Preferably step vii) is carried out in presence of hydrogen peroxide. It is also preferred to add a sulphite, such as sodium sulphite, when the reaction has reached completion in order to quench excess peroxide. It is also preferred to increase pH in step viii) by adding a strong base such as sodium hydroxide or potassium hydroxide. It is also preferred to recover 3-(5-methanesulfonyl-4-methyl-4H-1,2,4-triazol-3-yl)-pyridine in step ix) by filtration or centrifugation.

In a third aspect, the invention provides a process for the manufacture of the compound (1R)-1-[2-(3-methylphenyl)-2H-tetrazol-5-yl]ethanol of formula 13

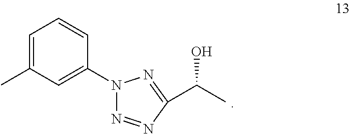

wherein
aa) (S)-2-methyl-CBS-oxaborolidine and borane or a borane complex, are dissolved in a suitable solvent; whereafter bb) the compound 1-[2-(3-methylphenyl)-2H-tetrazol-5-yl]ethanone of formula 12 is added to the mixture, providing the compound (1R)-1-[2-(3-methylphenyl)-2H-tetrazol-5-yl]ethanol of formula 13.

Preferably, the borane or borane complex in step aa)) is borane dimethylsulfide. Alternative borane sources such as borane tetrahydrofuran, borane trimethylamine and borane N,N-diethylaniline complexes may be used in the process. Preferably, said suitable solvent is tetrahydrofuran or 2-methyl tetrahydrofuran.

It is preferred that an excess of borane is quenched by adding an alcohol, such as methanol, after completion of formation of (1R)-1-[2-(3-methylphenyl)-2H-tetrazol-5-yl]ethanol.

Preferably, the compound (1R)-1-[2-(3-methylphenyl)-2H-tetrazol-5-yl]ethanol is recovered by extracting the reaction with an aqueous solution. Preferably, said aqueous solution is an aqueous solution of hydrochloric acid.

Alternatively, the compound (1R)-1-[2-(3-methylphenyl)-2H-tetrazol-5-yl]ethanol may be produced enzymatically by using an alcohol dehydrogenase and a co-factor selected from the group of NADH and NADPH, said co-factor being suitable for said alcohol dehydrogenase, comprising the steps of

AA) providing 1-[2-(3-methylphenyl)-2H-tetrazol-5-yl]ethanone and a suitable co-factor selected from the group of NADH and NADPH;
BB) dissolving said 1-[2-(3-methylphenyl)-2H-tetrazol-5-yl]ethanone and said suitable co-factor in a suspension of a lower alcohol and an aqueous buffer solution;
CC) adding a preparation of a suitable alcohol dehydrogenase and maintaining pH of the resulting mixture within the range 4.9-8.0;
DD) adding an organic solvent such as tert-butyl methylether; and
EE) recovering (1R)-1-[2-(3-methylphenyl)-2H-tetrazol-5-yl]ethanol from the organic phase.

In a preferred embodiment, said lower alcohol is isopropanol. It is also preferred that the buffer solution is an aqueous solution containing triethanolamine hydrochloride and magnesium chloride.

In a preferred embodiment, said alcohol dehydrogenase is an alcohol dehydrogenase referred to as IEP Ox58, manufactured by IEP GmbH, DE, and obtainable from DSM pharmaceutical products, Geleen, NL. However, any alcohol dehydrogenase (EC 1.1.1.1, CAS 9031-72-5) having NADH or NADPH as a co-factor and an ability to produce the (R) isomer of 1-[2-(3-methylphenyl)-2H-tetrazol-5-yl]ethanol could be used. Preferably, the enzyme should also be able to regenerate the co-factor that is consumed during reduction of 1-[2-(3-methylphenyl)-2H-tetrazol-5-yl]ethanone, by oxidizing isopropanol toacetone.

The organic solvent in step dd) is preferably tert-butyl-methylether but other ethers such as tetrahydrofuran, methyl tetrahydrofuran and diethyl ether can also be used.

Alternatively, (1R)-1-[2-(3-methylphenyl)-2H-tetrazol-5-yl]ethanol may be prepared by a asymmetric hydrogenation comprising the steps of:

1) adding said 1-[2-(3-methylphenyl)-2H-tetrazol-5-yl]ethanone to a suitable reaction medium containing a solvent and a catalytic amount of a transition metal based catalyst such as benzeneruthenium (II) chloride dimer in combination with S-Xyl-BINAP and S-DAIPEN or S-Xyl-Segphos in combination with S-DAIPEN or RR-Cyl-C*-Thunefos in combination with S-DAIPEN in the presence of a strong base such as potassium tert-butoxide in 2-propanol.

2) reacting 1-[2-(3-methylphenyl)-2H-tetrazol-5-yl]ethanon with hydrogen at an increased pressure.

### DETAILED DESCRIPTION OF THE INVENTION

Accordingly, the present invention provides a process of producing 3-{4-methyl-5-[(1R)-1-(2-(3-methylphenyl-2H-tetrazol-5-yl)-ethoxy]-4H-[1,2,4]triazol-3-yl}-pyridine. The process is divided into two separate branches leading to intermediates 3-(5-methanesulfonyl-4-methyl-4H-1,2,4-triazol-3-yl)-pyridine and (1R)-1-[2-(3-methylphenyl)-2H-tetrazol-5-yl]ethanol, respectively. In the final steps of the process these intermediates are allowed to react with each other leading to formation of 3-{4-methyl-5-[(1R)-1-(2-(3-methylphenyl-2H-tetrazol-5-yl)-ethoxy]-4H-[1,2,4]triazol-3-yl}-pyridine.

The synthesis of 3-(5-methanesulfonyl-4-methyl-4H-1,2,4-triazol-3-yl)-pyridine is outlined in Scheme 1 below:

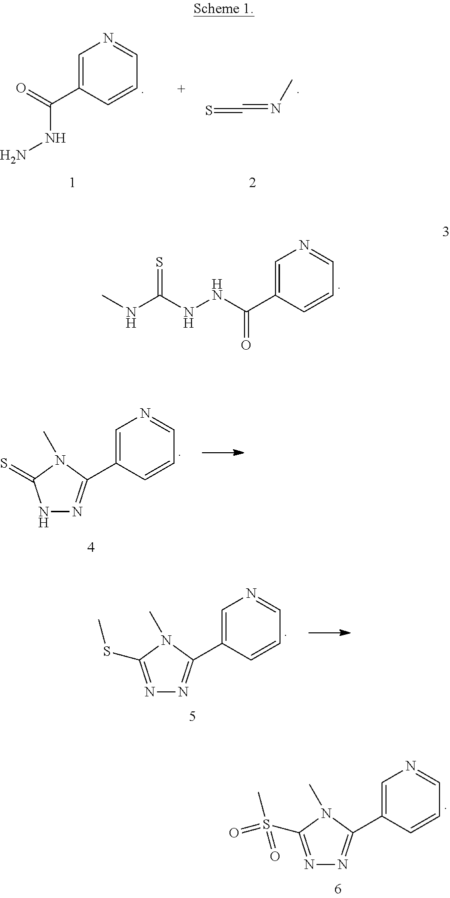

The process starts by adding nicotinic acid hydrazide (1) and an isothiocyanate such as methyl isothiocyanate (2) to a solvent selected from the group of water and a lower alcohol, such as n-butanol. When the reaction is completed, the resulting carbazide (3) is exposed to alkaline conditions without intermediate isolation leading to formation of 4-methyl-5-pyridin-3-yl-2,4-dihydro-3H-1,2,4-triazole-3-thione (4). Methyl iodide is then added to the resulting reaction without intermediate isolation and 3-[4-methyl-5-(methylthio)-4H-1,2,4-triazol-3-yl]-pyridine (5) is formed and isolated before next step.

In case water is used as solvent, sodium hydroxide is added to the reaction mixture containing carbazide (3) in order to increase pH. Sulfide (5) is isolated and purified by extraction with dichloromethane and subsequent precipitation in n-heptane. In a more preferred embodiment, n-butanol is used as solvent. In this case, pH of the carbazide (3)-containing reaction mixture is increased by adding tributylamine. Moreover, sulfide (5) is precipitated directly from the reaction mixture without addition of any anti-solvents and can be isolated by filtration.

Sulfide (5) is added to an aqueous sulfuric acid solution, where it reacts with a tungstate, such as sodium tungstate dihydrate. Preferably, the solution also contains hydrogen peroxide. When the reaction is completed, excess hydrogen peroxide is quenched by adding a bisulfite, such as sodium bisulfite. Then, pH of the resulting reaction medium is adjusted to pH 3-4 by adding an alkaline agent such as NaOH. The pH adjustment induces precipitation of 3-(5-methanesulfonyl-4-methyl-4H-1,2,4-triazol-3-yl)-pyridine (6).

The process for preparing (1R)-1-[2-(3-methylphenyl)-2H-tetrazol-5-yl]ethanol is disclosed in Scheme 2, below:

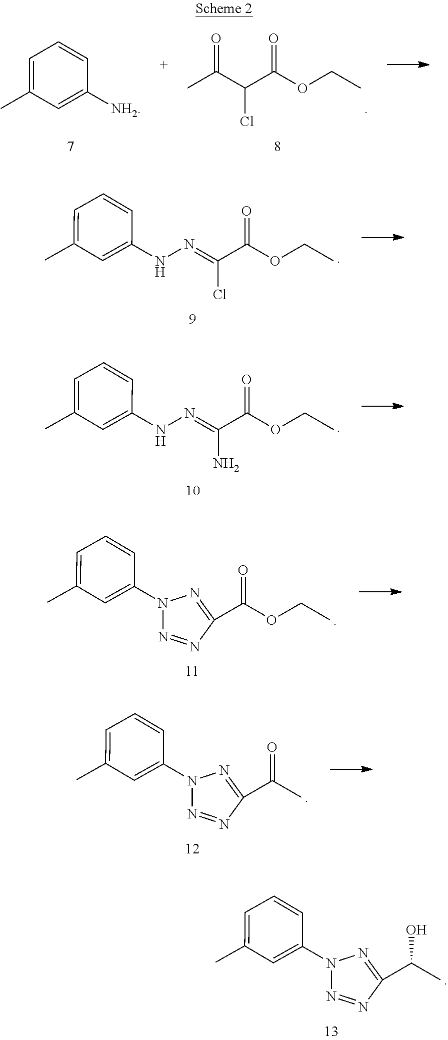

The first process steps up to formation of 1-[2-(3-methylphenyl)-2H-tetrazol-5-yl]-ethanone (12) have been disclosed in U.S. provisional application 60/981,294. m-Toluidine (7), sodium nitrite and sodium acetate is dissolved in a mixture of ethanol, water and hydrochloric acid at a temperature below 5° C. Ethyl 2-chloro-acetoacetate (8) is then added and allowed to react with m-toluidine (7) at room temperature. Resulting ethyl (2Z)-chloro-[(3-methylphenyl)-hydrazono]acetate (9) is obtained by extraction with 2-methyl tetrahydrofuran, and the methyl tetrahydrofuran phase is used as such in next step without intermediate isolation. An aqueous solution of ammonium hydroxide is brought into contact with the 2-methyl tetrahydrofuran phase leading to formation of ethyl (2Z)-amino-[(3-methylphenyl)-hydrazono]-acetate (10). The aqueous phase is discarded and intermediate compound (10) is precipitated from the 2-methyl tetrahydrofuran phase using n-heptane as an anti-solvent. Said ethyl (2Z)-amino-[(3-methylphenyl)-hydrazono]-acetate (10) is dissolved in a mixture of 2-methyl tetrahydrofuran and acetic acid and the resulting mixture is contacted with an aqueous solution of sodium nitrite, leading to formation of ethyl 2-(3-methylphenyl)-2H-tetrazole-5-carboxylate (11). The aqueous phase is discarded and a mixture of methyl magnesium bromide and triethylamine in toluene/tetrahydrofuran is added to the organic phase leading to formation of 1-[2-(3-methylphenyl)-2H-tetrazol-5-yl]-ethanone (12). The reaction mixture is quenched with acetic acid in 2-methyl tetrahydrofuran, and then washed with water and aqueous potassium carbonate solution. The water phase is discarded, the organic phase is concentrated and intermediate compound (12) is precipitated. Intermediate compound (12) is then added to a mixture of (S)-2-methyl-CBS oxaborolidine and borane or a borane complex and (1R)-1-[2-(3-methylphenyl)-2H-tetrazol-5-yl]ethanol (13) is recovered from the reaction.

Alternatively, 1-[2-(3-Methylphenyl)-2H-tetrazol-5-yl]-ethanone (12) and NADH are added to a mixture of a lower alcohol, such as isopropanol, and an aqueous buffer solution capable of maintaining pH in the range of 4.9-8.0. A preparation of an alcohol dehydrogenase (EC1.1.1.1, CAS 9031-72-5), preferably the preparation referred to as IEP Ox58, manufactured by IEP GmbH, DE and obtainable from DSM pharmaceutical products, Geleen, NL, is added and (1R)-1-[2-(3-methylphenyl)-2H-tetrazol-5-yl]ethanol (13) is formed. The product is recovered by extraction with an organic solvent, such as tert-butyl methyl ether or a similar ether, the aqueous phase is discarded and finally, the organic solvent is removed by evaporation.

The process of manufacturing 3-{4-methyl-5-[(1R)-1-(2-(3-methylphenyl-2H-tetrazol-5-yl)-ethoxy]-4H-[1,2,4]triazol-3-yl}-pyridine is illustrated in Scheme 3, below:

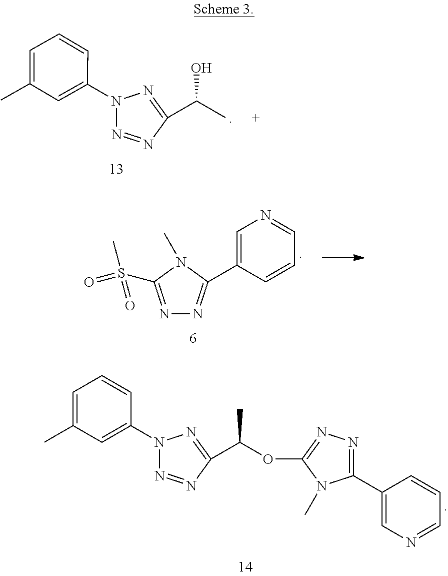

In the final step, 3-(5-methanesulfonyl-4-methyl-4H-1,2,4-triazol-3-yl)-pyridine and (1R)-1-[2-(3-methylphenyl)-2H-tetrazol-5-yl]ethanol is dissolved in tetrahydrofuran. Potassium tert-butoxide dissolved in tetrahydrofuran is added and the final product 3-{4-methyl-5-[(1R)-1-(2-(3-methylphenyl-2H-tetrazol-5-yl)-ethoxy]-4H-[1,2,4]triazol-3-yl}-pyridine (14) is formed. It is normally further purified, for instance by re-crystallization. A suitable solvent in this regard is isopropyl acetate.

### EXPERIMENTAL PART

The invention will now be disclosed with reference to the following examples. These examples are enclosed for information purposes and are not intended to restrict the scope of the invention.

### Reference Example 1

### Preparation of 1-[2-(3-methylphenyl)-2H-tetrazol-5-yl]-ethanone (compound 12)

m-Toluidine (6.86 g, 63.38 mmol) (7) was dissolved in ethanol (20 ml), water (7 ml) and 37% hydrochloric acid (13 ml, 158 mmol) and the solution was cooled to −5° C. A solution of sodium nitrite (4.96 g, 69.72 mmol) in water (14 ml) was added to the reaction mixture while keeping the reaction temperature below 5° C., then a solution of sodium acetate (15.60 g, 190.14 mmol) in water (31 ml) was added while keeping the reaction temperature below 0° C. Ethyl 2-chloroacetoacetate (10.87 g, 63.38 mmol) (8) was added and the reaction mixture was stirred overnight at 27° C. 2-Methyltetrahydrofuran (27 ml) was added and the temperature was adjusted to 40° C., the lower aqueous layer was discarded and the solution of ethyl (2Z)-chloro-[(3-methylphenyl)-hydrazono]acetate (9) was used as such in the next step.

Ammonium hydroxide (25% solution in water, 29 ml, 393 mmol) was added at 0° C. whereupon the mixture was warmed to 17° C. for 2 hours. The aqueous layer was discarded and 2-Methyltetrahydrofuran (14 ml) was added. The same volume solvent was distilled of at 50° C. under reduced pressure. The procedure was repeated using 21 ml 2-methyltetrahydrofuran and the solution was concentrated to 27 ml. The temperature was adjusted to 30° C. after which n-heptane (27 ml) was added and the solution was cooled to 5° C. during 4 hours whereon another portion of n-heptane (27 ml) was added to the resulting slurry. The product was isolated by filtration, washed with n-heptane (27 ml) and was dried at 40° C. under reduced pressure giving ethyl (2Z)-amino-[(3-methylphenyl)-hydrazono]-acetate (10), 11.19 g as a brown yellow powder in 72% yield over two steps.

A solution of Sodium Nitrite (3.94 g, 46.17 mmol) in water (22 ml) was added drop wise over 1 hour to a solution of ethyl (2Z)-amino-[(3-methylphenyl)-hydrazono]-acetate (10) (11.19 g, 48.70 mmol) in 2-methyltetrahydrofuran (112 ml) and acetic acid (11 ml, 185 mmol) held at 70° C. The solution was cooled to 35° C. and the aqueous phase was discarded. The organic phase was washed with water (22 ml) followed by potassium carbonate (15.95 g, 115.44 mmol) dissolved in water (45 ml). The organic phase was concentrated by 50% under reduced pressure at 50° C. The solution containing ethyl 2-(3-methylphenyl)-2H-tetrazole-5-carboxylate (11) was used as such in the following step.

To methyl magnesium bromide 1.4M in toluene/THF (3/1) (59.4 ml, 83.11 mmol) was added triethylamine (35 ml, 249 mmol) at ambient temperature. The mixture was cooled to −20° C. and was added drop wise while keeping the inner temp below −10° C. to the above solution of ethyl 2-(3-methylphenyl)-2H-tetrazole-5-carboxylate (11). The reaction mixture was quenched by adding the mixture to acetic acid (26 ml, 462 mmol) in 2-methyltetrahydrofuran (45 ml) while keeping the reaction temperature below 0° C. After complete addition, the mixture was warmed to 50° C. and the aqueous phase was discarded. The organic phase was washed with water (45 ml) followed by potassium carbonate (10.2 g, 73.9 mmol) dissolved in water (45 ml). The organic phase was concentrated to 20 ml under reduced pressure at 50° C. and isopropanol (60 ml) was added and the then cooled to 5° C. over 4 hours. The product was isolated by filtration, washed with cooled isopropanol (22 ml) and dried under reduced pressure at 40° C. giving 1-[2-(3-methylphenyl)-2H-tetrazol-5-yl]-ethanone (12), 5.46 g in 57% yield over two steps.

### Example 2

### Enzymatic reduction of 1-[2-(3-methylphenyl)-2H-tetrazol-5-yl]-ethanone to give (1R)-1-[2-(3-methylphenyl)-2H-tetrazol-5-yl]ethanol (compound 13)

A buffer solution was prepared comprising 100 mM triethanolamine and 2 mM magnesium chloride in water and pH was adjusted to 8.0 with aqueous NaOH.

20 g 1-[2-(3-Methylphenyl)-2H-tetrazol-5-yl]ethanone (12) and 12 mg NADH were mixed in 40 ml isopropanol and 50 ml buffer solution (see above) was added. 5 ml of enzyme preparation IEP Ox58 (manufactured by IEP GmbH and obtainable from DSM pharmaceutical products, Geleen, NL) was then added and the pH of the formed suspension is adjusted to 8.0 by addition of 1M NaOH. The suspension was stirred over night at 25° C. until complete conversion is obtained. The mixture is diluted with 375 mL of tert-butyl methylether and 125 mL of water are added. The mixture is stirred and the aqueous phase is discarded. The organic phase is washed 4 times with 125 mL 3 wt % NaCl in water. The organic phase is filtered through a bed of Hyflo and washed with 50 mL of tert-butyl methylether. The organic phase is concentrated in vacuum and 19.9 g, 99% of the wanted product is isolated as a brown oil.

### Example 3

### Preparation of 4-methyl-3-methylthio-5-(3-pyridyl)-1,2,4-triazole in water (compound 5)

Nicotinic acid hydrazid (900 g, 6.56 moles, 1 eq), Methyl isothiocyanate (480 g, 6.56 moles, 1 eq) and water (4.15 L, 5 rel. vol.) were charged to a 10 L reactor and the mixture was agitated at 60° C. until complete conversion was obtained. Sodium hydroxide (45% w/w) (700 g, 7.88 moles, 500 ml, 1.2 eq) was added during 30 min and the formed slurry went into solution, the solution was held at 60° C. for 2 h and was then cooled to 20 h. Methyl iodide (1123 g, 7.91 mol, 1.21 eq) was charged to the reaction mixture and full conversion was obtained after 1 h reaction time. Dichloromethan (2.7 L, 3 rel. vol.) was then charged and the mixture was agitated for 30 min. The organic layer was separated and the aqueous layer was washed twice with dichloromethane (2×2.7 L). The organic layers were combined and the aqueous layer was discarded. The organic phase was concentrated under reduce pressure at 40° C. to a total volume of 4 L and the temperature was adjusted to 0° C. n-Heptane (1.8 L, 2 rel. vol.) was charged during 2 h and the slurry was agitated for 8 h and a second portion of n-heptane (2.7 L, 3 rel. vol.) was charged. The slurry was aged for 1 h whereupon the product was isolated by filtration. The filter cake was washed twice with heptane (2×1 L) and the product was dried under reduced pressure at 40° C. giving 820 g, 64% with 95.8% w/w strength and a chromatographic purity of 98%.

### Example 4

### Preparation of 4-methyl-3-methylthio-5-(3-pyridyl)-1,2,4-triazole in n-butanol (compound 5)

To a warm solution of nicotinic acid hydrazide (80 g, 583 mmol) in n-Butanol (280 ml) was slowly added a solution of methyl isothiocyanate (43.5 g, 583 mmol) in n-Butanol (120 ml). The reaction mixture was held at 80° C. for 3 h for full conversion to the thiocarbazide. To the reaction mixture was tributylamine (171 ml, 700 mmol) added and the reaction mixture was held at 80° C. for 9 hours until full conversion to the thione was seen. The reaction mixture was cooled to 20° C. and methyl iodide was slowly added and the mixture was stirred for 1 hour. After full conversion to the sulfide the slurry was cooled to 0° C. over 4 hours and the precipitate was isolated by filtration, washed with isopropyl acetate (2*320 ml) and dried under reduced pressure to give 4-methyl-3-methylthio-5-(3-pyridyl)-1,2,4-triazole (91.6 g, 76% yield) as a white solid.

### Example 5

### Preparation of 4-methyl-3-methylsulfonyl-5-(3-pyridyl)-1,2,4-triazole (compound 6)

4-methyl-3-methylthio-5-(3-pyridyl)-1,2,4-triazole (10 g, 48.48 mmol) was mixed with sodium tungstate dihydrate (0.31 g, 0.97 mmol), water (40 mL) and sulfuric acid (2.63 mL, 48.48 mmol). The mixture was warmed to 50° C. and hydrogen peroxide (9.13 mL, 106.66 mmol) was added over 5 h. The solution was kept under stirring until completion when 39% sodium bisulfate in water (1.93 mL, 9.7 mmol) was added to quench excess peroxide. Water (40 mL) and methanol (10 mL) were added followed by the addition of 45% NaOH (4.33 mL, 82.42 mmol) during 30 minutes. The resulting clear solution was seeded with 4-methyl-3-methylsulfonyl-5-(3-pyridyl)-1,2,4-triazole (100 mg) and was held at 50° C. for 1 hour when 45% sodium hydroxide (0.39 mL, 7.27 mmol) was charged over 30 minutes. The mixture was held at 50° C. for further 5 hours and was then cooled to 10° C. over 10 hours. The product was isolated and was washed with water (20 mL) followed by isopropanol (20 mL) giving 4-methyl-3-methylsulfonyl-5-(3-pyridyl)-1,2,4-triazole (8.16 g, 70% yield) as a white solid.

### Example 6

### Preparation of 3-{4-methyl-5-[(1R)-1-(2-(3-methylphenyl-2H-tetrazol-5-yl)-ethoxy]-4H-[1,2,4]triazol-3-yl}-pyridine (compound 14)

To 4-methyl-3-methylsulfonyl-5-(3-pyridyl)-1,2,4-triazole (12.2 g, 51.0 mmol) and (1R)-1-[2-(3-methylphenyl)-2H-tetrazol-5-yl]ethanol (10.0 g, 48.6 mmol) mixed in tetrahydrofuran (40 mL) at 25° C., was added a solution of 1M potassium tert-butoxide in tetrahydrofuran (51 mL, 48.6 mmol) over 1 hour. The reaction mixture was held until consumption of starting materials is complete and the mixture was quenched by the addition of a solution of sodium chloride (6.0 g) and 37% hydrochloric acid (0.4 mL) dissolved in water (30 mL). The aqueous phase was discarded and the organic phase was washed two times with further NaCl-solution (6.0 g in 30 ml water). The organic phase was concentrated to ca 3 relative volumes and isopropyl acetate (80 mL) was added and the solution was concentrated to ca 3 relative volumes. Isopropyl acetate (70 mL) was added, the temperature adjusted to 70° C. and the solution was filtered to remove solid impurities. The solution was cooled to 50° C. and was seeded with the title compound (10mg 0.1w/w %). The solution was held at 50° C. for 1 hour and then cooled to 10° C. over 5 hours. The product was isolated and washed with isopropyl acetate (20 mL) and dried under reduced pressure at 40° C. to give 3-{4-methyl-5-[(1R)-1-(2-(3-methylphenyl-2H-tetrazol-5-yl)-ethoxy]-4H-[1,2,4]triazol-3-yl}-pyridine (14.4 g, 80% yield).

### Example 7

### Non-enzymatic preparation of (1R)-1-[2-(3-methylphenyl)-2H-tetrazol-5-yl]ethanol) (compound 13)

(S)-2-Methyl-CBS-oxaborolidine (16.3 mL (16.3 mmol, 1M solution in toluene) and borane dimethylsulfide (9.25 mL 97.5 mmol) were mixed and diluted with 18 mL 2-is methyltetrahydrofuran. The resulting solution was heated to 45° C. A solution of 1-[2-(3-methylphenyl)-2H-tetrazol-5-yl]-ethanone, (32.8 g, 162.3 mmol) dissolved in 395 mL 2-methyltetrahydrofurane was added to the CBS-borane solution over approximately 3.5 h. The reaction had reached complete conversion after the addition of the ketone solution. The inner temperature was then set to 15° C. and 41 mL methanol was added to quench excess borane. The quenched reaction mixture was then extracted with 42 mL 6M HCl. The temperature was adjusted to 25° C. after which 115 ml water was added, the phases was separated and the aqueous phase was discarded. The organic phase was extracted with 120 ml water, the phases allowed to separate and the aqueous phase was discarded and the organic phase concentrated under reduced pressure, giving 31.7 g of the wanted product (1R)-1-[2-(3-methylphenyl)-2H-tetrazol-5-yl]ethanol) with 84% ee.

### Example 8

### Preparation of 1-[2-(3-methylphenyl)-2H-tetrazol-5-yl)]-ethanone (compound 12) using a flow reactor

The reaction was performed in an ART PR37 equipped with a PL37/3-12 plate from Alfa Laval.

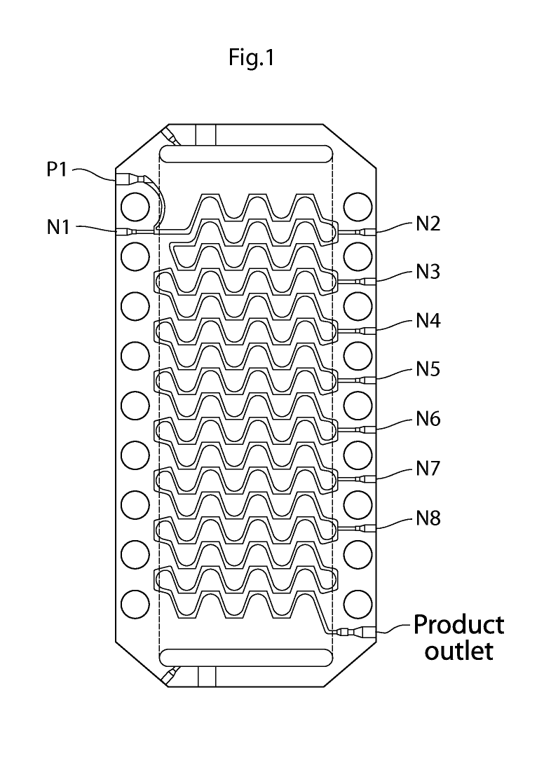

FIG. 1 shows how each of the solutions 1, 2 and 3 respectively as described below, are fed into the ART PR37 equipped with a PL37/3-12 plate. P1, and N1-N8 are inlet ports. The final product is collected at the outlet “collection”.

Three starting solutions were used:

### Solution 1:

Ethyl 2-(3-methylphenyl)-2H-tetrazole-5-carboxylate (27.0 g, 115.0 mmol, 12.4 wt %), triethylamine (56.1 ml, 402.5 mmol, 18.9 wt %) and 2-methyltetrahydrofuran (68.7 wt %) was injected at Port P1 at a flow rate of 15.2 g/min.

### Solution 2

1.4 M Methyl magnesium bromide solution (98.6 ml, 138.0 eq) was injected at Port N1 at a flow rate of 7.1 g/min

### Solution 3:

Acetic acid (308 ml, 1.79 mol, 36.1 wt %), 2-methyltetrahydrofuran (29.5 wt %), water (34.4 wt %) was injected at Port N8 at a flow rate of 21.6 g/min

The pressure was set at 17 bar and the mantle temperature was at 0° C. This provided a temperature at port N2 of 10° C. The reaction solution was collected during approximately 5 minutes where ca 9.6 g, 41.2 mmol Ethyl 2-(3-methylphenyl)-2H-tetrazole-5-carboxylate had been reacted. The reaction solution was analyzed by HPLC at 254 nm and provided the following results:

1-[2-(3-methylphenyl)-2H-tetrazol-5-yl)]-ethanone 84.3 area %, tert-alcohol by-product 13 area % and aldol by-product 2.4 area %.

The aqueous phase was discarded and the organic phase was washed with water (50 ml) followed pH adjustment to pH 7 with saturated potassium carbonate solution. The aqueous phase was discarded and the organic phase was concentrated to 28 g under reduced pressure. To the concentrate was added isopropanol (74 ml) and the mixture was warmed to 55° C. resulting in a clear solution, which was cooled to 20° C. during 3 hours. The product was isolated by filtration, washed with isopropanol (25 ml) and dried under reduced pressure at 40° C., providing 1-[2-(3-methylphenyl)-2H-tetrazol-5-yl)]-ethanone 6.45 g, 76% yield.

### Example 9

### Catalytic enantioselective hydrogenation of 1-[2-(3-methylphenyl)-2H-tetrazol-5-yl]-ethanon to give (1R)-1-[2-(3-methylphenyl)-2H-tetrazol-5-yl]-ethanol

Under an inert atmosphere 8 mM benzeneruthenium (II) chloride dimer (65 μl, 0.5 μmol) in dimethylformamide was mixed with 27.5 mM 1-[2-(3-methylphenyl)-2H-tetrazol-5-yl]-ethanon (43 μl) in toluene/tetrahydrofuran (10:6), R-Xyl-BINAP (0.7 mg, 1.0 μmol) and dimethylformamide (100 μl). The mixture was agitated at 100° C. for 1 hour and was then cooled to 30° C. To the mixture was added 22 mM R-DAIPEN (45.5 μl, 1.0 μmol) in toluene. The mixture was agitated at 30° C. for 1 hour when potassium tert-butoxide in isopropanol (450 μl, 5 mg/ml) was added. After 2 minutes 1-[2-(3-methylphenyl)-2H-tetrazol-5-yl]-ethanon (20.0 mg, 98.9 μmol) was added and the reaction mixture was pressurized to 50 bar hydrogen pressure and was agitated for 1 hour. The reaction was sampled showing formation of (1S)-1-[2-(3-methylphenyl)-2H-tetrazol-5-yl]-ethanol in 99% enantioselectivity.

The combination of R-Xyl-Segphos and R-DAIPEN provided 99% enantioselectivity of (1S)-1-[2-(3-methylphenyl)-2H-tetrazol-5-yl]-ethanol.

The combination of R ax, SS-Xyl-C*-Thunefos and R-DAIPEN provided 99% enentioselectivity of (1S)-1-[2-(3-methylphenyl)-2H-tetrazol-5-yl]-ethanol.

The combination of CTH-C3-Thunefos and R,R-DACH provided 98% enentioselectivity of (1S)-1-[2-(3-methylphenyl)-2H-tetrazol-5-yl]-ethanol.

The combination of R-Xyl-BINAP and R-DPEN provided 97% enentioselectivity of (1S)-1-[2-(3-methylphenyl)-2H-tetrazol-5-yl]-ethanol.

The combination of R-Xyl-BINAP and R,R-DACH provided 97% enentioselectivity of (1S)-1-[2-(3-methylphenyl)-2H-tetrazol-5-yl]-ethanol.

The combination of R-Xyl-Segphos and R-DPEN provided 97% enentioselectivity of (1S)-1-[2-(3-methylphenyl)-2H-tetrazol-5-yl]-ethanol.

The use of the other enantiomer of the catalysts will provide (1R)-1-[2-(3-methylphenyl)-2H-tetrazol-5-yl]-ethanol.

1. A process for the manufacture of the compound 3-{4-methyl-5-[(1R)-1-(2-(3-methylphenyl-2H-tetrazol-5-yl)-ethoxy]-4H-[1,2,4]triazol-3-yl}-pyridine of formula 14: 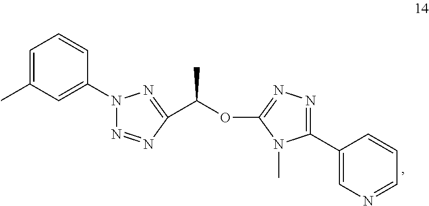
wherein the compound 3-(5-methanesulfonyl-4-methyl-4H-1,2,4-triazol-3-yl)-pyridine of formula 6: 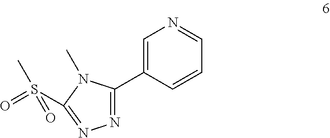
and the compound (1R)-1-[2-(3-methylphenyl)-2H-tetrazol-5-yl]ethanol of formula 13: 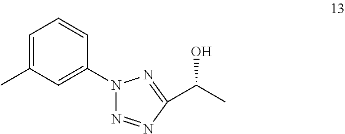
are dissolved in an aprotic solvent, whereafter a base is added, providing the compound of formula 14.

2. A process according to claim 1, wherein the aprotic solvent is tetrahydrofuran, 2-methyltetrahydrofuran, DMSO, acetonitrile, sulfolan, or isopropyl acetate.

3. A process according to claim 1, wherein the base is an alkoxide base.

4. A process according to claim 3, wherein the alkoxide base is lithium-tert-butoxide, sodium-tert-butoxide, potassium-tert-butoxide, lithium-tert-amylate, sodium-tert-amylate, or potassium-tert-amylate.

5. A process according to claim 1, wherein the base is a hydride base.

6. A process according to claim 5, wherein the hydride base is sodium or potassium hydride.

7. A process according to claim 1, wherein the compound of formula 14 is purified by crystallization from isopropyl acetate.

8. A process for the manufacture of the compound 4-methyl-3-methylthio-5-(3-pyridyl)-1,2,4-triazole of formula 5: 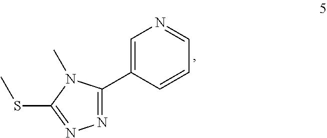
wherein the method comprises:
i) dissolving nicotinic acid hydrazide in a first solvent;
ii) adding an isocyanate to the solution of step until complete conversion is obtained;
iii) adding a base to the resulting mixture of step ii, providing the compound 4-methyl-5-pyridin-3-yl-2,4-dihydro-3H-1,2,4-triazole-3-thione of formula 4: 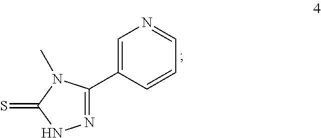
and
iv) charging methyl iodide to the reaction mixture of step iii, providing the compound of formula 5,
wherein steps i-iv) are carried out without intermediate isolation.

9. A process according to claim 8, wherein the first solvent is n-butanol or water.

10. A process according to claim 8, wherein the isocyanate is methyl isocyanate.

11. A process according to claim 8, wherein the base is sodium hydroxide, potassium hydroxide, or tributylamine.

12. A process according to claim 8, wherein the first solvent is n-butanol, the isocyanate is methyl isocyanate, and the base is tributylamine.

13. A process according to claim 8, wherein the following steps are carried out after step iv:
v) dissolving the compound of formula 5 in an acid aqueous solution;
vi) adding a tungstate to react with the compound 4 of formula 5; and
vii) increasing the pH of the reaction mixture, resulting in the precipitation of the compound of formula 4, which is thereafter recovered.

14. A process according to claim 13, wherein the tungstate is sodium tungstate dihydrate

15. A process according to claim 13, wherein the acid aqueous solution is a diluted sulfuric acid solution.

16. A process according to claim 13, wherein:
step vii is carried out in presence of hydrogen peroxide;
a sulfite is added when the reaction has reached completion in order to quench excess peroxide;
pH is increased in step vii by adding a strong base such as sodium hydroxide or potassium hydroxide; and
the compound of formula 5 is recovered by filtration.

17. A process of providing the compound (1R)-1-[2-(3-methylphenyl)-2H-tetrazol-5-yl]ethanol of formula 13: 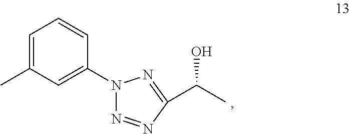
wherein the process comprises:
aa) (S)-2-methyl-CBS-oxaborolidine and borane or a borane complex are dissolved in a suitable solvent; and
bb) 1-[2-(3-methylphenyl)-2H-tetrazol-5-yl]ethanone is added to the mixture providing the compound of formula 13.

18. A process according to claim 17, wherein:
the borane or borane complex in step aa is borane dimethylsulfide, borane tetrahydrofuran, borane trimethylamine, and borane N,N-diethylaniline complexes, and
the suitable solvent is tetrahydrofuran or 2-methyl tetrahydrofuran.

19. A process according to claim 18, wherein an excess of borane is quenched by adding an alcohol, after completion of formation of (1R)-1-[2-(3-methylphenyl)-2H-tetrazol-5-yl]ethanol.

20. A process according to claim 19, wherein the alcohol is methanol.

21. A process according to claim 17, wherein (1R)-1-[2-(3-methylphenyl)-2H-tetrazol-5-yl]ethanol is recovered by extracting the reaction with an aqueous solution.

22. A process according to claim 21, wherein the aqueous solution is an aqueous solution of hydrochloric acid.

23. A process according to claim 17, wherein (1R)-1-[2-(3-methylphenyl)-2H-tetrazol-5-yl]ethanol is further purified by crystallization from a solvent or solvent mixture selected from the group of aromatic hydrocarbons, ethers, alkanes, and polar aprotic solvents, as single crystallization solvent or in any combination, with or without water present.
